(19) United States
(12) Patent Application Publication
(10) Pub. No.: US 2008/0044396 A1
(43) Pub. Date: Feb. 21, 2008

(54) Method of Raising Livestock

(75) Inventors: Shuichi Shiomi, Yokohama-shi (JP); Shinichi Kanou, Yokohama-shi (JP)

Correspondence Address: SMITH, GAMBRELL & RUSSELL, 1130 CONNECTICUT AVENUE, N.W., SUITE 1130, WASHINGTON, DC, 20036, US

(73) Assignee: PLOBAIO INTERNATIONAL CO., LTD.

(21) Appl. No.: 11/741,203

(22) Filed: Apr. 27, 2007

(30) Foreign Application Priority Data
Jun. 28, 2006 (JP) 2006-177560

(51) Int. Cl. A61K 35/00; A61P 43/00

(52) U.S. Cl. 424/93.45

(57) ABSTRACT

The present invention provides a method of raising livestock, which can improve an efficiency of propagating or rearing without using a drug such as an antibiotic or a disinfectant. The method uses a lactic acid bacterium (such as Enterococcus faecium or Lactobacillus fermentum) proliferated and cultured in a medium containing a nutrient source derived from a plant. Specifically, a uterus of a domestic animal after delivery is washed with a lavage fluid containing the lactic acid bacterium as an active ingredient; the lactic acid bacterium is orally administered to a baby domestic animal suffering from diarrhea during the lactation period; and tap water containing the lactic acid bacterium is sprayed over the interior of the livestock house to condition the humidity. Thus, the lactic acid bacterium is totally used in breeding and growing, whereby healthy condition of a domestic animal is maintained, resulting in being capable of producing well-grown meet with good quality and good taste. The meat is safe because it has no adverse affect on the human body due to a residual harmful substance or the like.

### BACKGROUND OF THE INVENTION

1. Field of the Invention

The preset invention relates to a method of raising livestock such as pig or cow using a lactic acid bacterium (including a wash of the inside of uterus).

2. Description of the Related Art

In recent years, there have been problems such as side effects caused by administration of a drug such as an antibiotic or a disinfectant to domestic animals, weakening of the immune system by killing beneficial bacteria, emergence of resistant bacteria, and adverse affects of harmful materials remaining in meats of domestic animals to the human body when the domestic animals are supplied as edible meats.

On the other hand, the inventors of the present invention have proposed that addition of an additive for domestic animal feeds containing Lactobacillus fermentum classified into lactic acid bacteria to the feeds is effective for maintaining healthy conditions of domestic animals without reducing or administering a drug such as an antibiotic as disclosed in JP 2005-124433 A and JP 2005-198536 A.

However, it is important for management of livestock farming not only to maintain the healthy conditions of domestic animals but also to reduce a drug such as an antibiotic or a disinfectant in various cases involved in propagating or rearing. For example, if the inside of uterus or the like after delivery becomes inflamed, the uterus is washed with an iodine-based disinfectant (such as Isodine (trademark)). The washing may cause sore of uterine wall, low fever, and stress due to the symptoms. Instead of disinfectant, physiological saline may be used but cannot achieve a sufficient effect in the lavage of the uterus, and physiological saline itself is instantly contaminated.

The inventors of the present application have made extensive studies by examining whether or not a lactic acid bacterium with excellent properties as an additive for domestic animal feeds can be used for various cases involved in propagating or rearing of the domestic animals. As a result, there has discovered a method of raising livestock (including a wash of the inside of uterus) described below in detail.

### SUMMARY OF THE INVENTION

The present invention have been made to solve the problems described above, and an object of the present invention is to provide a method of raising livestock (including a wash of the inside of uterus), which can be carried out without using a drug such as an antibiotic or a disinfectant, and can improve the efficiency of propagating or rearing.

To solve the above-mentioned problems, according to an aspect of the present invention, there is provided a method of raising livestock, including washing an inside of uterus of a domestic animal after delivery with a lavage fluid, the lavage fluid containing as an active ingredient a lactic acid bacterium which has been proliferated and cultured in a medium containing a nutrient source derived from a plant.

In the method of raising livestock, it is preferable that a concentration of the lactic acid bacterium in the lavage fluid be 1.2×10⁷ or more cells/ml.

In the method of raising livestock, in addition to the wash of the inside of uterus, it is preferable that the method further include orally administering the lactic acid bacterium to a baby domestic animal suffering from diarrhea during a lactation period. In this case, it is preferable that a number of the lactic acid bacterium to be orally administered be 1.2×10¹⁰ or more cells per day.

In the method of raising livestock, in addition to the wash of the inside of uterus, it is preferable that the method further include: adding the lactic acid bacterium to tap water in a livestock house; and supplying the tap water as drinking water.

In the method of raising livestock, in addition to the wash of the inside of uterus, it is preferable that the method further include: adding the lactic acid bacterium to tap water in a livestock house; and spraying the tap water over an interior of the livestock house.

In the method of raising livestock, it is preferable that the method further include: producing a lactic acid bacterium powder for raising livestock; and using the lactic acid bacterium powder for applying the lactic acid bacterium to the domestic animal. In the method, the lactic acid bacterium powder is produced by: forming a culture that is obtained by proliferating and culturing a lactic acid bacterium in a medium containing a nutrient source derived from a plant into a slurry state; spraying and drying the slurry-state culture in a hot-air drying chamber to form powder thereof; and delivering the powder from the hot-air drying chamber to a powder accumulation chamber that is conditioned at a temperature lower than that of the hot-air drying chamber via a delivery pipe by a hot air.

In the method of raising livestock, it is preferable that the lactic acid bacterium include one kind or more kinds of lactic acid bacteria belonging to the genus Enterococcus or Lactobacillus.

According to another aspect of the present invention, there is provided a method of raising livestock, including orally administering a lactic acid bacterium to a baby domestic animal suffering from diarrhea during the lactation period, the lactic acid bacterium having been proliferated and cultured in a medium containing a nutrient source derived from a plant.

In the method of raising livestock, it is preferable that a number of the lactic acid bacterium to be orally administered be 1.2×10¹⁰ or more cells per day.

In the method of raising livestock, it is preferable that the method further include: producing a lactic acid bacterium powder for raising livestock; and using the lactic acid bacterium powder for applying the lactic acid bacterium to the domestic animal. In the method, the lactic acid bacterium powder is produced by: forming a culture that is obtained by proliferating and culturing a lactic acid bacterium in a medium containing a nutrient source derived from a plant into a slurry state; spraying and drying the slurry-state culture in a hot-air drying chamber to form powder thereof; and delivering the powder from the hot-air drying chamber to a powder accumulation chamber that is conditioned at a temperature lower than that of the hot-air drying chamber via a delivery pipe by a hot air.

In the method of raising livestock, it is preferable that the lactic acid bacterium include one kind or more kinds of lactic acid bacteria belonging to the genus Enterococcus or Lactobacillus.

According to still another aspect of the present invention, there is provided a method of raising livestock, including: adding a lactic acid bacterium to tap water in a livestock house, the lactic acid bacterium having been proliferated and cultured in a medium containing a nutrient source derived from a plant; and supplying the tap water as drinking water.

In the method of raising livestock, it is preferable that the method further includes spraying the tap water over an interior of the livestock house.

According to the method of raising livestock of the present invention, the efficiency of propagating or rearing can be improved without using a drug such as an antibiotic or a disinfectant. The method as described above includes: washing an inside of a uterus of a domestic animal after delivery with a lavage fluid, the lavage fluid containing as an active ingredient a lactic acid bacterium which has been proliferated and cultured in a medium containing a nutrient source derived from a plant; orally administering the lactic acid bacterium to a baby domestic animal suffering from diarrhea during a lactation period; or adding the lactic acid bacterium to tap water in a livestock house.

### BRIEF DESCRIPTION OF THE DRAWINGS

In the accompanying drawings:

FIG. 1 is a photograph showing a result of an experiment on antiproliferation effect of Lactobacillus fermentum against Escherichia coli.

FIG. 2 schematically shows a part of manufacturing equipment for a lactic acid bacterium powder for raising livestock to be used in a method of raising livestock according to an embodiment of the present invention.

FIG. 3 is a drawing showing a wash of the inside of uterus in the method of raising livestock according to the embodiment of the present invention.

FIG. 4 is a photograph showing a result of an experiment on antiproliferation effect of Lactobacillus fermentum against Clostridium perfringens.

### DETAILED DESCRIPTION OF THE INVENTION

Hereinafter, the best mode for carrying out the present invention will be described with reference to the drawings. In an embodiment of the present invention, a description will be made of a breeding method in a case where a domestic animal to be bred is a pig, but it goes without saying that the method can be applied to other domestic animals than pig (such as cow, horse, sheep, goat, and chicken). Meanwhile, in the present invention, the term “raising livestock” includes all things related to propagating or rearing of the domestic animals.

First, a wash of the inside of uterus of a mother pig according to a method of raising livestock of the present invention will be described. In general, crossing in propagation of pigs are carried out at an age of about eight months, and the pigs deliver baby pigs after the lapse of about 114 days of pregnancy. Moreover, after a lapse of about 40 days from delivery, the crossing is performed again. Thus, the parturition is repeated about 6 times in about three years. The wash of the uterus was carried out after each the parturition.

In washing of the inside of uterus according to a method of raising livestock of the present invention, the uterus is washed by injecting a lavage fluid containing as an active ingredient a lactic acid bacterium which has been proliferated and cultured in a medium containing a nutrient source derived from a plant (hereinafter, referred to as “plant-derived lactic acid bacterium”). The fetal and placental residues with rich nutrients remain at the inside of uterus, an upper portion of an oviduct, and a vagina after the delivery, where bacteria serving as malignant bacteria or pathogenic bacteria (such as Escherichia coli and Staphylococcal bacteria) are easy to grow. The injected lavage fluid dissolves such the fetal and placental residues, and are naturally discharged from the body via the vagina after a given period of time. After the wash of the uterus, the plant-derived lactic acid bacterium adheres to the nutritious uterus, the upper portion of the oviduct, and the vagina; and prevents bacteria, which enter from an outside and serve as malignant bacteria or pathogenic bacteria, from growing by an antiproliferative effect to thereby promote a recovery of the uterus. Note that, in the present invention, the medium containing a plant-derived nutrient source is preferably a medium containing a nutrient source prepared from a plant (such as soybean powder or starch) in an amount at least larger than that of a nutrient source prepared from animals.

Mother pigs that have been subjected to the wash of the uterus with the plant-derived lactic acid bacterium may deliver many healthy pigs and exhibit estrus at earlier. In the case where artificial insemination is carried out for the pigs, the implantation rates are high. Meanwhile, the mother pigs have little stress and breast-feed baby pigs smoothly, so the baby pigs are well grown.

Hereinafter, the plant-derived lactic acid bacterium will be described in detail.

First, the antiproliferative effect of the plant-derived lactic acid bacterium will be described. Experiments by the inventors of the present invention revealed that the plant-derived lactic acid bacterium has the antiproliferative effect on various bacteria serving as malignant bacteria or pathogenic bacteria (such as Escherichia coli and Staphylococcal bacteria). The plant-derived lactic acid bacterium suppresses proliferation of bacteria serving as malignant bacteria or pathogenic bacteria by the lactic acid bacterium itself or a product thereof (such as lactic acid). If the proliferation is suppressed, the bacteria do not act as malignant bacteria or pathogenic bacteria. The lactic acid bacteria used in the experiments are Enterococcus faecium belonging to the genus Enterococcus and Lactobacillus fermentum belonging to the genus Lactobacillus.

Enterococcus faecium being a plant-derived lactic acid bacterium has a coccal form, generates no gas from glucose, and grows under alkaline (pH 9.6) conditions and acidic (pH 4.8) conditions. It has high resistance to bile. In addition, it is low-temperature-resistant and can live at 4° C. The plant-derived lactic acid bacterium, Lactobacillus fermentum, is in a bacillus form and generates a gas from glucose. It has resistance to acid and can live under strong acidic (pH 2.0) conditions. Moreover, it is high-temperature-resistant and can live at 60° C. for 30 minutes.

The experiment method is as follows. First, 1 g of a lactic acid bacterium powder for raising livestock including Enterococcus faecium or Lactobacillus fermentum (lactic acid bacterium powder for raising livestock will be described below in detail) was dissolved in 10 ml of physiological saline to prepare a sample solution (the number of cells: about 0.9×10⁹ cells/ml). Next, a piece of water-absorbent filter paper R_{A}, which is cut into a round shape with a diameter of 4 cm and is absorbed with the sample solution, is placed onto the center of a petri dish S_{H} of MRS agar medium. Then, a suspension including 1×10⁷ cells of a test bacterium (Escherichia coli or Staphylococcal bacteria) is spread to the petri dish S_{H} and left at 37° C. for 72 hours, followed by observation. Note that the water-absorbent filter paper used is one manufactured by ADVANTEC (No.26-WA, code:02481180).

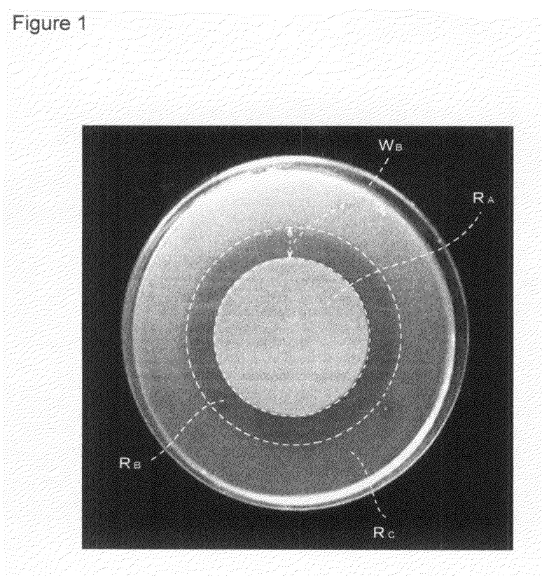

FIG. 1 is a photograph showing the results of the experiment for Escherichia coli and Lactobacillus fermentum. There is a region R_{B} (with a width of W_{B}) between the water-absorbent filter paper R_{A} placed at the center of the petri dish S_{H} and the region R_{C} of the test bacterium, containing the test bacterium proliferated in a very small amount. That is, the plant-derived lactic acid bacterium present in the water-absorbent filter paper R_{A} was found to suppress proliferation of the test bacterium. Note that also in the cases of Escherichia coli and Enterococcus faecium, Staphylococcal bacteria and Enterococcus faecium, and Staphylococcal bacteria and Lactobacillus fermentum, the plant-derived lactic acid bacteria were confirmed to suppress proliferation of the test bacteria.

As described above, the plant-derived lactic acid bacteria have the antiproliferative effect on various bacteria serving as malignant bacteria or pathogenic bacteria. Meanwhile, the plant-derived lactic acid bacteria are tough to survive under circumstances such as, inside the uterus or in an instrument for use in washing the uterus, when compared to a lactic acid bacterium that has been proliferated and cultured in a medium containing nutrient sources primarily derived from animal products such as milk and meat. Meanwhile, at least one, that is, one or two or more of the plant-derived lactic acid bacteria may be present in combination. If a bacillus is combined with a coccus, the bacillus and coccus intermingle with each other, thereby increasing their density. Meanwhile, if Enterococcus faecium and Lactobacillus fermentum having different properties are combined, they complement each other and extend the temperature and pH ranges applicable to all the plant-derived lactic acid bacteria, resulting in increasing availability.

Next, a method of producing a lavage fluid to be used for washing the uterus of a domestic animal will be described. The steps in the method are broadly divided into a step of producing a lactic acid bacterium powder for raising livestock (lactic acid bacterium powder) from an inoculum of a lactic acid bacterium and a step of producing a lavage fluid from the lactic acid bacterium powder for raising livestock.

The step of producing a lactic acid bacterium powder for raising livestock can be further divided into a pure culture step, a proliferation/culture step, a slurry step, and a powderization step.

In the pure culture step, glucose, yeast extract, and peptone are mixed in purified water and sterilized by heating in an autoclave, and an inoculum of a lactic acid bacterium (in the case where several lactic acid bacteria are used in combination, a mixture of all inoculums) is added thereto and cultured in an incubator (for example, at 40° C. for 48 hours). Thus, a pure culture medium of a lactic acid bacterium is yielded. Note that the inoculum is supplied in the form of a bacterial solution or a lactic acid bacterium powder for raising livestock for the pure culture.

The proliferation/culture step is a step of proliferating/culturing a bacterium by mixing purified water, the above-described pure culture medium, and various nutrients serving as a plant-derived medium with stirring. Specifically, 1.5% the above-described pure culture medium, 9% defatted soybean powder, 6% starch, 1% (black) molasses, 4% skim milk, 0.5-1.5% natural salt, 0.5% sodium glutamate, 0.1% amino acid, 0.1% inulin, and 0.2% trehalose are mixed in 100% purified water, and the mixture is incubated at 40° C. for 48 hours to proliferate a plant-derived lactic acid bacterium. Note that the percentages refer to ratios by weight (the same applies to the following description).

The slurry step is a step of forming the above mixture into a slurry state by using a mixer.

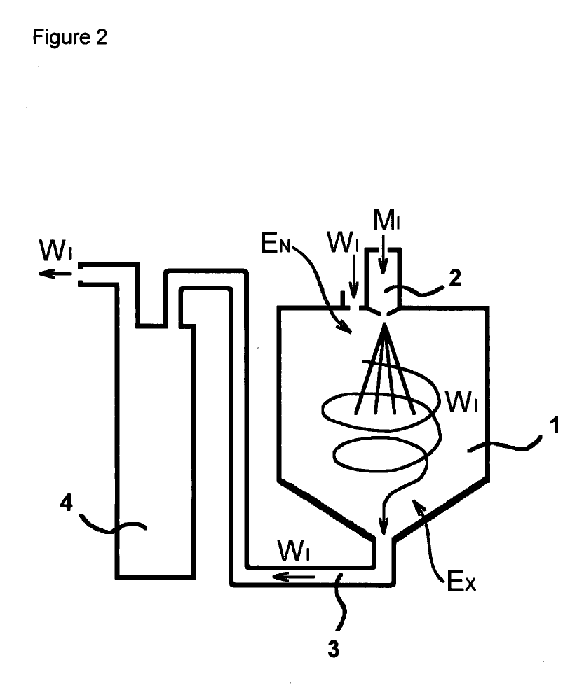

The powderization step is a step of powderizing the slurry-like mixture by spray drying using a spray drier. Specifically, as shown in the schematic drawing of manufacturing equipment of FIG. 2, the equipment includes a drying chamber 1. Then, a nozzle 2 and a delivery pipe 3 are attached to the top and bottom of the drying chamber 1, respectively. The delivery pipe 3 extends not only laterally but also upward, and the other end of the pipe is attached to a powder accumulation chamber 4 juxtaposed to the drying chamber 1. The temperature of an entry E_{N} of hot air W₁ in the drying chamber 1 is 150 to 180° C., and the temperature of an exit Eₓ is about 100° C. The revolution rate (caused by rotation of a motor (not shown)) is set to about 4,000 rpm. In the drying chamber 1, the above-described mixture M₁ is sprayed from the nozzle, and rapid water removal (drying) is carried out with the hot air W₁ revolving at a high wind speed, to thereby yield a powder. The powder is delivered to the powder accumulation chamber 4 with a temperature (about 40° C.) lower than that of the drying chamber 1 via the delivery pipe 3 on stream of the hot wind W₁. Thus, rapid drying in the drying chamber 1 and rapid cooling in the powder accumulation chamber 4 can significantly suppress damage or death of bacteria due to high temperature. Also, with the protective membrane of starchy materials and sugars formed on the outer surface during the drying step, influences of high temperature on the viable bacteria inside the protective membrane can be suppressed.

The powderization step is carried out as described above, to thereby yield a sufficiently dried (water content: about 3% or less) lactic acid bacterium powder for raising livestock with a small average particle size. The viable count of plant-derived lactic acid bacteria in the lactic acid bacterium powder for raising livestock is high and is 1.2×10¹⁰−1.2×10¹¹ cells/g in terms of Enterococcus faecium and Lactobacillus fermentum. In addition, the lactic acid bacterium powder for raising livestock is sufficiently dried, and viable bacteria inside the protective membrane of starchy materials and sugars is protected, so that the bacteria can be stored for a long period of time (for example, 10-20 years in a low humidity ambient environment).

The lavage fluid to be used for washing the uterus of a domestic animal is produced by adding the lactic acid bacterium powder for raising livestock to a liquid medium. The liquid medium is a solution prepared by adding salt to purified water at a concentration of about 10%. For example, the lavage fluid is produced by adding the lactic acid bacterium powder for raising livestock to the solution at a concentration of about 2%. As a result, 200 ml of the lavage fluid contains 4.8×10¹⁰ or more cells of a plant-derived lactic acid bacterium. The experiment by the inventors of the present application revealed that an effect of lavage of the uterus can be obtained if 200 ml of a lavage fluid contains 2.4×109 or more cells of a plant-derived lactic acid bacterium. That is, if the concentration of the plant-derived lactic acid bacterium in the lavage fluid is 1.2×10⁷ or more cells/ml, the plant-derived lactic acid bacterium plays a role as an effective ingredient.

As described above, the lavage fluid can be produced by adding a lactic acid bacterium powder for raising livestock before each wash of the uterus of a domestic animal, so that the plant-derived lactic acid bacterium can be easily used. Note that the lavage fluid itself has an antiproliferative effect on various bacteria serving as malignant bacteria or pathogenic bacteria, so that it can be stored (for about two weeks), and therefore is highly convenient.

Next, the method of washing the uterus of a domestic animal will be described.

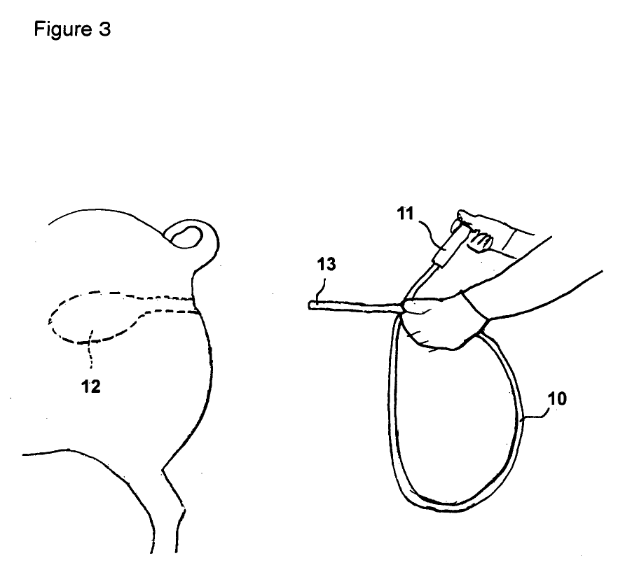

As shown in FIG. 3, a lavage fluid 11 (about 200 ml) is injected into a uterus 12 of a domestic animal after delivery that lies down by using the rubber, plastic, or metallic tubular instrument 10 (such as catheter). An insertion part 13 of the instrument was designed so as to be suitable for the structures of the uterus, cervix uteri, etc. and to cause no damage to them. The injection may be carried out by a gravitational dropped injection or an electronically driven injection. The lavage fluid injected into the uterus is naturally discharged one or two hours after the injection of the uterus.

As described above, the wash of the uterus of a domestic animal in a method of raising livestock of the present invention requires no large and expensive apparatus and is easy to be performed. In addition, the washing is carried out without using an iodine-based disinfectant. As a result, the uterus can be washed without worrying about an adverse affect of residual harmful substances and the like on the human body when the domestic animal is supplied as edible meat. Note that the lavage fluid discharged from the animal after washing the uterus contains collagen in a large amount and contains no drug such as an iodine-based disinfectant, so that the solution can be used in various fields (such as cosmetics).

Table 1 below shows the results of the experiment on next births in the case where the wash the inside of uterus was carried out for all domestic animals by a method of raising livestock of the present invention with a lavage fluid containing a plant-derived lactic acid bacterium as described above. Meanwhile, Table 2 below shows the results of the experiment in the case where lavage of the uterus was carried out for all domestic animals with an iodine-based disinfectant (conventional method). These experiments were carried out at the same hog farm.

TABLE 1

| | | | | | | Average | Average |
|---|---|---|---|---|---|---|---|
| | Number of | | | Number of | | birth | weaning |
| | healthy | Number of | Number of | crushing | Number of | weight | weight |
| | pigs | weak pigs | stillbirths | deaths | parturition | (kg) | (kg) |
| 1 | 13 | | | | 3 | 1.61 | 8.00 |
| 2 | 12 | | | | 2 | 1.45 | 9.00 |
| 3 | 13 | | | | 3 | 1.76 | 10.00 |
| 4 | 13 | | | | 3 | 1.48 | 9.50 |
| 5 | 12 | | | | 2 | 1.50 | 8.00 |
| 6 | 13 | | | | 3 | 1.47 | 9.00 |
| 7 | 11 | | | | 2 | 1.63 | 9.50 |
| 8 | 13 | | | | 3 | 1.78 | 9.00 |
| 9 | 15 | | | | 2 | 1.05 | 9.50 |
| 10 | 13 | | | | 4 | 1.50 | 9.00 |
| 11 | 12 | | | | 3 | 1.45 | 8.50 |
| 12 | 13 | | | | 5 | 1.71 | 10.00 |
| 13 | 12 | | | | 5 | 1.50 | 9.00 |
| 14 | 13 | | | | 6 | 1.78 | 9.50 |
| 15 | 11 | | | | 6 | 1.45 | 10.00 |
| 16 | 13 | | | | 7 | 1.70 | 10.00 |
| 17 | 12 | | | | 7 | 1.40 | 9.50 |
| 18 | 13 | | | | 8 | 1.80 | 9.00 |
| 19 | 12 | | | | 8 | 1.50 | 9.50 |
| 20 | 13 | | | | 6 | 1.80 | 10.50 |
| 21 | 13 | | | | 5 | 1.75 | 10.00 |
| 22 | 13 | | | | 5 | 1.65 | 9.50 |
| 23 | 12 | | | | 6 | 1.50 | 9.00 |
| Average | 12.6 | 0.0 | 0.0 | 0.0 | 4.5 | 1.57 | 9.33 |

TABLE 2

| | | | | | | | Average |
|---|---|---|---|---|---|---|---|
| | Number of | | | Number of | | Average | weaning |
| | healthy | Number of | Number of | crushing | Number of | birth weight | weight |
| | pigs | weak pigs | stillbirths | deaths | parturition | (kg) | (kg) |
| 1 | 11 | | 2 | 1 | 3 | 1.72 | 8.00 |
| 2 | 11 | | | | 2 | 2.20 | 8.00 |
| 3 | 6 | | 3 | | 7 | 1.16 | 5.00 |
| 4 | 13 | | 3 | | 4 | 1.81 | 5.90 |
| 5 | 15 | | | | 3 | 1.26 | 5.50 |
| 6 | 13 | | 1 | | 3 | 1.61 | 6.00 |
| 7 | 13 | | 1 | | 5 | 1.76 | 7.00 |
| 8 | 9 | 1 | 1 | | 6 | 2.00 | 7.00 |
| 9 | 15 | | | | 3 | 0.93 | 5.00 |
| 10 | 13 | | | | 9 | 1.05 | 6.00 |
| 11 | 10 | | | | 8 | 1.55 | 9.70 |
| 12 | 10 | | | | 2 | 1.98 | 8.60 |
| 13 | 13 | 2 | | | 3 | 1.48 | 6.10 |
| 14 | 11 | | | | 6 | 1.52 | 6.80 |
| 15 | 10 | 1 | | | 5 | 1.51 | 6.50 |
| 16 | 10 | | 2 | | 9 | 1.90 | 6.50 |
| 17 | 12 | | | | 6 | 1.45 | 6.50 |
| 18 | 13 | | | | 2 | 1.46 | 6.50 |
| 19 | 15 | | | | 4 | 1.23 | 6.50 |
| Average | 11.7 | 0.2 | 0.7 | 0.05 | 4.7 | 1.56 | 6.69 |

According to the results of these experiments, the number of healthy pigs in the case where the wash the inside of uterus was carried out by the method of raising livestock of the present invention is larger than that in the case of the conventional method. Meanwhile, in the latter case, the ratios of appearance of the weak pigs and stillbirths were quite large, while in the former case, weak pigs and stillbirths were not observed. The results clarified that the wash of the uterus by the method of raising livestock of the present invention recovered the uteruses of the mother pigs before implantation.

Moreover, there was no crushing death of baby pigs in the case of washing of the uterus by the method of raising livestock of the present invention, while crushing deaths of baby pigs occurred in the case of the conventional method. Meanwhile, the average birth weights of the baby pigs in both cases are almost the same, but the average weaning weights of the baby pigs are significantly different, and baby pigs grew better in the case of the method of raising livestock of the present invention. If the method of raising livestock of the present invention is not used, the mother pigs tend to lack affection for baby pigs due to side effects (such as sore of uterine wall and low fever) caused by washing of the uterus with an iodine-based disinfectant, and therefore they are unwilling to breast-feed baby pigs or crush baby pigs to death. On the other hand, in the case of the method of raising livestock of the present invention, the mother pigs had not only no inflammation and the like but also no stress, and they breast-fed the baby pigs smoothly. Moreover, the vaginas of the mother pigs were closed in two or three days, and the uteruses recovered early, and the pigs exhibited estrus within five days after weaning.

Next, growing of baby pigs in the method of raising livestock of the present invention will be described.

In general, baby pigs are more likely to develop diarrhea during the lactation period at the age of 15-35 days when antibodies transferred from mother pigs by colostrum intake decrease, and the baby pigs have high death rates (for example, about 30%) even if antibiotics are injected. In the method of raising livestock of the present invention, a plant-derived lactic acid bacterium is mixed in a product to be taken and orally administered to a baby domestic animal suffering from diarrhea during the lactation period. Thus, the diarrhea is ameliorated without using antibiotics.

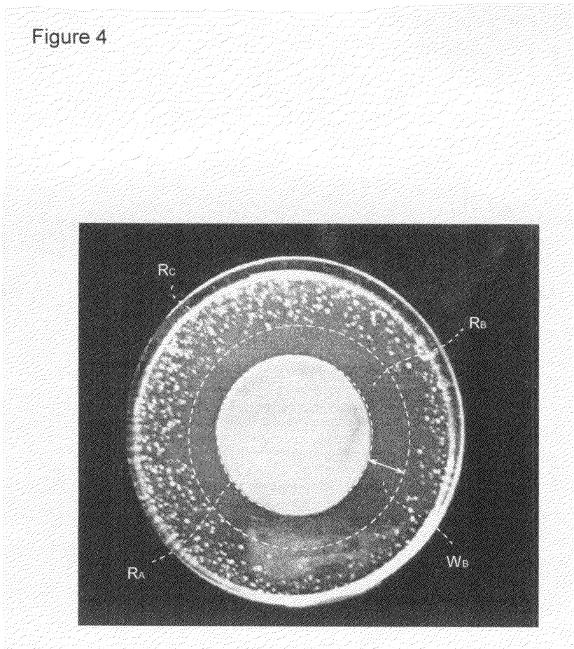

As described above, the plant-derived lactic acid bacteria have antiproliferative effects on various bacteria serving as malignant bacteria or pathogenic bacteria. The results of the experiments of the antiproliferative effects on bacteria such as Escherichia coli and Staphylococcal bacteria, which are easy to proliferate at the uterus and the like of a domestic animal after delivery, were described above with reference to FIG. 1 and the like. The experiments for bacteria such as Clostridium perfringens, Serratia bacteria, and Salmonella bacteria, which may cause diseases in pigs, were also carried out in the same way as above. FIG. 4 is a photograph showing the results of the experiments of Lactobacillus fermentum for Clostridium perfringens, and the bacterium was found to have antiproliferative effect.

Also, the other bacteria were confirmed to have antiproliferative effects. Meanwhile, the plant-derived bacteria have good resistance to acids when compared to lactic acid bacteria that has been proliferated and cultured in a medium primarily containing a nutrient prepared from an animal, therefore a large number of (about 7- to 8-fold) the plant-derived bacteria reach the bowels.

In the case where 1 g or more per day (more specifically, 1 to 2 g) of a lactic acid bacterium powder for raising livestock, that is, 1.2×10¹⁰ or more cells per day of a plant-derived lactic acid bacterium was mixed in artificial milk or the like in a dish and orally administered to the baby pigs suffering from diarrhea shown in Table 1 above, the pigs recovered from diarrhea in one or two days. Meanwhile, the number of baby pigs that were dead before weanling was found to be zero.

When a plant-derived lactic acid bacterium is added to a feed and administered to pigs during the weaning or fattening period, infection or illness caused by pathogenic bacteria can be prevented. Specifically, a mixture is obtained by adding a lactic acid bacterium powder for raising livestock to the used residues of pulse and the like. Then the mixture is allowed to ferment and added to a feed.

Next, conditioning of a livestock house (piggery) in the method of raising livestock of the present invention will be described.

In the method of raising livestock using a plant-derived lactic acid bacterium and without using a drug such as an antibiotic or a disinfectant, it is desirable to condition piggeries as described below to relieve stress and make pleasant environments. In a piggery, a breeding piggery, a parturition piggery, a weaning piggery, and a shipping piggery are arranged in this order, and pigs are grown in the respective piggeries depending on growing stages of pigs.

Moreover, regulation of the temperature and humidity in a piggery provides an effect of making a comfortable environment for pigs and is effective for plague measures. For example, the air-heat conditioning in the wintertime can prevent poor development of baby pigs and reduce mortality. Regulation of humidity can prevent bronchitis and prevent loss of body strength. Specifically, for baby pigs, the temperature and humidity are regulated to 20-25° C. and 65-70% in the wintertime, or 25 to 30° C. and 60 to 70% in the summertime, while for middle-aged pigs, the temperature and humidity are regulated to 15 to 20° C. and 60 to 70% in the wintertime, or 25 to 30° C. and 70 to 75% in the summertime. For adult pigs, the temperature and humidity are regulated to 10 to 20° C. and 65 to 70% in the wintertime, or 25 to 30° C. and 70 to 75% in the summertime. In particular, in a breeding piggery, it is effective to maintain the temperature and humidity to a certain values and to hang curtains on the sides of the piggery to prevent invasion of contagious diseases.

The humidity of a piggery is regulated by spraying tap water. In this process, if a solution obtained by adding a plant-derived lactic acid bacterium to tap water in the piggery is supplied as drinkable water and sprayed in the piggery, the drinkable water can prevent infection or illness caused by pathogenic bacteria as is the case with the above-described feed, and adjust the humidity, and prevent dermatosis by adhesion to the skins of pigs with the plant-derived lactic acid bacterium in sprayed water. For example, water containing 0.1% lactic acid bacterium powder for raising livestock may flow in a water pipe in the piggery.

As described above, the method of raising livestock of the present invention, where a plant-derived lactic acid bacterium, in particular, a lactic acid bacterium powder for raising livestock is totally used in breeding and growing, is performed without using a drug such as an antibiotic or a disinfectant, so it is environmentally friendly and enables safe breeding of domestic animals without adverse affects on the human body caused by residual harmful substances when the animals are supplied as edible meats. Meanwhile, the method can maintain healthy conditions of pigs and produce well-grown meet with good quality and good taste.

Note that the present invention is not limited to the above-described embodiments and can be modified within the scope of the claims in various ways. For example, it goes without saying that specific values of the production conditions, environmental conditions, and the like, or the materials and the like can be appropriately altered or added.

What is claimed is:

1. A method of raising livestock, comprising washing an inside of uterus of a domestic animal after delivery with a lavage fluid, the lavage fluid containing as an active ingredient a lactic acid bacterium which has been proliferated and cultured in a medium containing a nutrient source derived from a plant.

2. A method of raising livestock according to claim 1, wherein a concentration of the lactic acid bacterium in the lavage fluid is 1.2×10⁷ or more cells/ml.

3. A method of raising livestock according to claim 1, further comprising orally administering the lactic acid bacterium to a baby domestic animal suffering from diarrhea during a lactation period.

4. A method of raising livestock according to claim 3, wherein a number of the lactic acid bacterium to be orally administered is 1.2×10¹⁰ or more cells per day.

5. A method of raising livestock according to claim 1, further comprising: adding the lactic acid bacterium to tap water in a livestock house; and supplying the tap water as drinking water.

6. A method of raising livestock according to claim 1, further comprising: adding the lactic acid bacterium to tap water in a livestock house; and spraying the tap water over an interior of the livestock house.

7. A method of raising livestock according to claim 1, further comprising: producing a lactic acid bacterium powder for raising livestock; and using the lactic acid bacterium powder for applying the lactic acid bacterium to the domestic animal,
wherein the lactic acid bacterium powder is produced by:
forming a culture that is obtained by proliferating and culturing a lactic acid bacterium in a medium containing a nutrient source derived from a plant into a slurry state;
spraying and drying the slurry-state culture in a hot-air drying chamber to form powder thereof; and
delivering the powder from the hot-air drying chamber to a powder accumulation chamber that is conditioned at a temperature lower than that of the hot-air drying chamber via a delivery pipe by a hot air.

8. A method of raising livestock according to claim 3, further comprising: producing a lactic acid bacterium powder for raising livestock; and using the lactic acid bacterium powder for applying the lactic acid bacterium to the domestic animal,
wherein the lactic acid bacterium powder is produced by:
forming a culture that is obtained by proliferating and culturing a lactic acid bacterium in a medium containing a nutrient source derived from a plant into a slurry state;
spraying and drying the slurry-state culture in a hot-air drying chamber to form powder thereof; and
delivering the powder from the hot-air drying chamber to a powder accumulation chamber that is conditioned at a temperature lower than that of the hot-air drying chamber via a delivery pipe by a hot air.

9. A method of raising livestock according to claim 5, further comprising: producing a lactic acid bacterium powder for raising livestock; and using the lactic acid bacterium powder for applying the lactic acid bacterium to the domestic animal,
wherein the lactic acid bacterium powder is produced by:
forming a culture that is obtained by proliferating and culturing a lactic acid bacterium in a medium containing a nutrient source derived from a plant into a slurry state;
spraying and drying the slurry-state culture in a hot-air drying chamber to form powder thereof; and
delivering the powder from the hot-air drying chamber to a powder accumulation chamber that is conditioned at a temperature lower than that of the hot-air drying chamber via a delivery pipe by a hot air.

10. A method of raising livestock according to claim 6, further comprising: producing a lactic acid bacterium powder for raising livestock; and using the lactic acid bacterium powder for applying the lactic acid bacterium to the domestic animal,
wherein the lactic acid bacterium powder is produced by:
forming a culture that is obtained by proliferating and culturing a lactic acid bacterium in a medium containing a nutrient source derived from a plant into a slurry state;
spraying and drying the slurry-state culture in a hot-air drying chamber to form powder thereof; and
delivering the powder from the hot-air drying chamber to a powder accumulation chamber that is conditioned at a temperature lower than that of the hot-air drying chamber via a delivery pipe by a hot air.

11. A method of raising livestock according to claim 1, wherein the lactic acid bacterium comprises one kind or more kinds of lactic acid bacteria belonging to the genus Enterococcus or Lactobacillus.

12. A method of raising livestock according to claim 3, wherein the lactic acid bacterium comprises one kind or more kinds of lactic acid bacteria belonging to the genus Enterococcus or Lactobacillus.

13. A method of raising livestock according to claim 5, wherein the lactic acid bacterium comprises one kind or more kinds of lactic acid bacteria belonging to the genus Enterococcus or Lactobacillus.

14. A method of raising livestock according to claim 6, wherein the lactic acid bacterium comprises one kind or more kinds of lactic acid bacteria belonging to the genus Enterococcus or Lactobacillus.

15. A method of raising livestock, comprising orally administering a lactic acid bacterium to a baby domestic animal suffering from diarrhea during the lactation period, the lactic acid bacterium having been proliferated and cultured in a medium containing a nutrient source derived from a plant.

16. A method of raising livestock according to claim 15, wherein a number of the lactic acid bacterium to be orally administered is 1.2×10¹⁰ or more cells per day.

17. A method of raising livestock according to claim 15, further comprising: producing a lactic acid bacterium powder for raising livestock; and using the lactic acid bacterium powder for applying the lactic acid bacterium to the domestic animal,
wherein the lactic acid bacterium powder is produced by:
forming a culture that is obtained by proliferating and culturing a lactic acid bacterium in a medium containing a nutrient source derived from a plant into a slurry state;
spraying and drying the slurry-state culture in a hot-air drying chamber to form powder thereof; and
delivering the powder from the hot-air drying chamber to a powder accumulation chamber that is conditioned at a temperature lower than that of the hot-air drying chamber via a delivery pipe by a hot air.

18. A method of raising livestock according to claim 16, wherein the lactic acid bacterium comprises one kind or more kinds of lactic acid bacteria belonging to the genus Enterococcus or Lactobacillus.

19. A method of raising livestock, comprising: adding a lactic acid bacterium to tap water in a livestock house, the lactic acid bacterium having been proliferated and cultured in a medium containing a nutrient source derived from a plant; and supplying the tap water as drinking water.

20. A method of raising livestock according to claim 19, further comprising spraying the tap water over an interior of the livestock house.
